(19) United States
(12) Patent Application Publication
(10) Pub. No.: US 2004/0081714 A1
(43) Pub. Date: Apr. 29, 2004

(54) Cosmetic and/or pharmaceutical preparations

(76) Inventors: Gilles Pauly, Nancy (FR); Philippe Moser, Essey-Les-Nancy (FR); Louis Danoux, Saulxures Les Nancy (FR); Olga Freis, Seichamps (FR); Florence Henry, Villiers-Les-Nancy (FR); Muriel Pauly-Florentiny, Nancy (FR); Anne Guezennec, Croissy Sur Seine (FR); Joelle Guesnet, Bures Sur Yvette (FR)

Correspondence Address: COGNIS CORPORATION, PATENT DEPARTMENT, 300 BROOKSIDE AVENUE, AMBLER, PA, 19002, US

(21) Appl. No.: 10/250,870

(22) PCT Filed: Jan. 5, 2002
(86) PCT No.: PCT/EP02/00053

(30) Foreign Application Priority Data
Jan. 15, 2001 (FR) 01/00492

(51) Int. Cl. A61K 35/78

(52) U.S. Cl. 424/774

(57) ABSTRACT

A cosmetic or pharmaceutical composition containing an extract of a resurrection plant.

### FIELD OF THE INVENTION

This invention relates generally to the field of cosmetics and, more particularly, to preparations with an effective content of extracts of resurrection plants and to the use of the extracts and the active substances present therein for the production of the preparations.

### PRIOR ART

A key reason for the ageing of skin is the loss of water from the upper layers of the epidermis and the wrinkling associated therewith. Accordingly, one of the ways cosmetic chemists seek to counter this phenomenon is to provide active substances which counteract environmental stress and dehydration and/or which have a protective function so that the cells are fortified in their ongoing struggle against environmental poisons. To this end, occasionally unusual pathways have to be followed to find a solution. Thus, it may be appropriate to gather important information from the knowledge with which nature provides us and to apply it to meet particular needs.

In the desert regions and arid zones of Africa, Asia and America, a number of plant families have developed a remarkable tolerance to drought which enables them to withstand up to 98% dehydration over a period of one year without damage and thereafter to regenerate themselves completely and to form flowers within 24 hours of the first monsoon rains. These poikilohydric representatives are known collectively as resurrection plants and include mosses, lichens and ferns and a number of flowering plants (angiospermae) of which studies have shown that the anatomical, biochemical and physiological adaptation is attributable to the genome.

During the drought phase, the plants are exposed to two different stresses, i.e. on the one hand mechanical stress and, on the other hand, oxidative stress. Resurrection plants have a number of ways of avoiding mechanical stress, of which shrinkage and the sharing of vacuoles to reduce stress on the plasma membrane are generally widespread. Other effects include the increased incorporation of xyloglucans and methylesters of pectin in the cell wall and the accumulation of osmolytes or osmoregulating molecules (for example sucrose, mannitol, D-ononitol, trehaloses, fructans, amino acids, etc.), so that the cell wall is strengthened and the production of toxic metabolites during dehydration is suppressed.

In addition, the interruption of cell respiration and photosynthesis during the drought phase leads to the formation of free radicals which are capable of damaging proteins, fats and nucleic acids. To prevent this, pigments of the anthocyan type and special enzymes are increasingly encountered in the cells, including for example superoxide dismutase, gutathione reductase and ascorbate peroxidase, which engage in the oxidative metabolism and are known as natural radical trappers.

The molecular fundamentals of tolerance to drought have not yet been fully elucidated. However, according to investigations conducted by D. Bartels at Bonn University, it seems clear that plant hormones, such as abscisic acid (ABA) for example, induce tolerance to drought. Since those investigations, a number of genes involved both in the process of desiccation and in rehydration have also been isolated. It was surprisingly found that those genes are homologous to genes that are also found in embryos of ripening seeds. For example, the gene dsp-22 (desiccation stress protein) is activated in the event of desiccation and stimulates the formation of a 21 KDa protein which accumulates in the chloroplasts [cf. D. Bartels et al., EMBO Journal, 11(8), 2771 (1992)]. In addition, changes in the metabolism of sugars are of importance. For example, the leaves of unstressed plants show high concentrations of the unusual sugar 2-octulose which is converted during desiccation into sucrose and appears to perform a protective function in the process. The process is reversible on rehydration. Reference is also made in this connection to International Patent Application WO 97/42327 (University of Mexico) which reports on the isolation of a gene from the resurrection plant Selaginella lepidophylla which produces the sugar trehalose-6-phosphate.

Accordingly, the problem addressed by the present invention was to provide new active substances with which, in general terms, the skin and hair could be protected from environmental influences and, more particularly, the skin could be prevented from drying out. In addition, the skin and hair would be afforded additional protection against osmotic and temperature-induced shock.

### DESCRIPTION OF THE INVENTION

The present invention relates to cosmetic and/or pharmaceutical preparations containing extracts of resurrection plants.

It has surprisingly been found that the extracts—which are also known as survival fractions—or the active substances present therein, which are mainly osmolytes (polysaccharides), terpenes, antioxidants and phytohormones and also proteins, solve the problem stated above in excellent fashion. The extracts may be used as such although individual constituents may also be isolated from them and then mixed in a different composition according to requirements.

Resurrection Plants

Resurrection plants are not a coherent group but can be found in very different plant families, among which the families of the Poacea, Scrophulariacea, Myrothamnacea and/or Velloziacea are mentioned above all.

In one particular embodiment of the invention, the preparations contain extracts of resurrection plants selected from the group of the botanical families of the Poacea, Scrophulariacea, Myrothamnacea and/or Velloziacea.

The most important representatives of the Poaceae include the genus Spirobolus, for example a grass which grows to a height of 60 to 120 cm and develops pink-colored flowers. It occurs above all on the American continent, especially in Costa Rica, where the species Spirobolus cubensis, Spirobolus indicus, Spirobolus heterotepsis, Spirobolus capillaris, Spirobolus flexuosus, Spirobolus cryptandrus and Spirobolus airoides can be found. A particularly important example of a resurrection plant from the family of the Scrophulariaceae is the genus Craterostigma, more particularly the species Craterostigma plantigineum. From the family of the Myrothamnaceae, mention is made above all of Myrothamnus niedenzu and Myrothamnus flabellifolia. According to the invention, particular preference is attributed to the family of the Myrothamnus flabellifolia which was described for the first time in 1891 by Engler and Pranti. This plant is a flat shrub which does not shed its leaves in the dry winter months, but applies them flat against the branches and comes back to life with the first summer rains. Key constituents of the extracts of its leaves are arbutin, anthocyans, polysaccharides (sucrose, glucose, trehalose, fructose, glucosyl-9-glycerol) and phytohormones (for example abscisic acid); terpenes such as, for example, carvones and perillic alcohol can also be found. Like octulose, arbutin also plays an important, albeit different, role in resistance to drought because, as a hydroquinone source, it prevents the peroxidation of unsaturated lipids in the cell membranes. Typical examples of resurrection plants from the Velloziacea family are the representatives of the genus Xerophyta, such as for example the Xerophyta retinervis and Xerophyta viscosa native to Madagascar which are flat bushes that develop magnificent violet flowers in the monsoon season. Extracts of plants of the geni Boea, Ramonda, Hamelea, Chamaegigas and Selaginella such as, for example, Selaginella lepidophylla and survival fractions of protein-rich angiospermous or gymnospermous plants or microorganisms such as, for example, Saccharomyces cerevisiae are also suitable for the purposes of the invention.

Extraction

The extracts may be prepared in known manner, i.e. for example by aqueous, alcoholic or aqueous/alcoholic extraction of the plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found for example in Hagers Handbuch der pharmazeutischen Praxis (5th Edition, Vol. 2, pp. 1026-1030, Springer Verlag, Berlin-Heidelberg-New York 1991). The percolation method is advantageous for industrial application. Fresh plants or parts thereof are suitable as the starting material although dried plants and/or plant parts which may be mechanically size-reduced before extraction are normally used. Any size reduction methods known to the expert, such as freeze grinding for example, may be used. Suitable solvents for the extraction process are organic solvents, water (preferably hot water with a temperature above 80° C. and, in particular, above 95° C.) or mixtures of organic solvents and water, more particularly low molecular weight alcohols with more or less large water contents. Extraction with distilled or nondistilled water, methanol, ethanol and aqueous solutions of thse two alcohols is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the active principles of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionaly be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried leaves are in the range from 3 to 20 and more particularly 6 to 10% by weight. The present invention includes the observation that the extraction conditions and the yields of the final extracts may be selected by the expert according to the desired application. These extracts, which generally have active substance contents (=solids contents) of 0.5 to 10% by weight, may be used as such although the solvent may also be completely removed by drying, more particularly by spray drying or freeze drying. The extracts may also be used as starting materials for the preparation of the pure active substances where they cannot be produced more simply and inexpensively by the synthetic route.

Active Substances

Instead of the extracts, the active substances present in the survival fractions may also be used individually or in the form of mixtures. They may be products obtained by purifying the extracts or by synthetic routes. The products obtainable from the extracts according to the invention by purification are particularly preferred. Typical examples of suitable active substances are osmolytes (for example octulose, sucrose, glucose, trehalose, fructose, glucosyl-9-glycerol, xyloglucans, methyl esters of pectins), terpenes (for example carvones, perillic alcohol), antioxidants (for example arbutin, anthocyans, superoxide dismutase, glutathione reductase, ascorbate peroxidase) and phytohormones (for example abscisic acid). In one particular embodiment of the invention, the preparations contain extracts with effective contents of osmolytes, terpenes, antioxidants and/or phytohormones.

Octulose, arbutin, abscisic acid and mixtures thereof are particularly important in this regard. The extracts are used in effective quantities, i.e. in concentrations of 0.001 to 1 and preferably 0.01 to 0.1% by weight (based on the amount of active substance and the final preparation), depending on the quantity of active substances (solids) present therein. The quantities mentioned apply accordingly to the pure active substances.

Commercial Applications

The present invention also relates to the use of extracts of resurrection plants for the production of cosmetic and/or pharmaceutical preparations and as active substances

for regulating the water metabolism in the skin or for regulating skin moisture

for strengthening the cell metabolism for protection against harmful environmental influences, more particularly for protecting the cells against such environmental influences as heat shock, cold shock or osmotic shock,

for protecting the skin and hair against damage by UV radiation,

for protecting the skin and hair against free radicals and

for protecting the macromolecules in the skin cells and cell membranes.

Finally, the present invention also relates to the use of octulose, arbutin and/or abscisic acid for the production of cosmetic and/or pharmaceutical preparations.

Cosmetic and/or Pharmaceutical Preparations

The extracts or active principles may be may be used for the production of cosmetic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, perservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysor-bates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

Ethylene Oxide Addition Products

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations from DE 20 24 051 PS.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerol-3-Diisostearate (Lameform® TGI), Polyglyceryl-4 Isostearate (Isolan® GI 34), Polyglyceryl-3 Oleate, Diisostearoyl

Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 moles of ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat poly-peptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

Besides the extracts according to the invention and the effective contents of active substances in these extracts as active substances against damage by UV radiation, other UV protection factors may also be used.

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

Biogenic agents in the context of the invention are, for example, tocopherol, tocopherol acetate, tocopherol palmitate, ascorbic acid, deoxyribonucleic acid and fragmentation products thereof, β-glucans, retinol, bisabolol, allantoin, phytantriol, panthenol, AHA acids, amino acids, ceramides, pseudoceramides, essential oils, plant extracts and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils and Aromas

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture was a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

### EXAMPLES

Example 1. 30 g dried leaves or stems of Myrothamnus flabellifolia were coarsely crushed in a mortar and then transferred to a glass reactor where 300 ml distilled water wer poured on. The infusion was heated to ca. 80° C. and extracted with stirring for 1 hour at that temperature. The mixture was then cooled to 20° C. and centrifuged for 15 mins. at a speed of 5000 G. The supernatant liquid was separated from the residue by filtration (mesh width of filter 0.45 μm), giving 190 ml of extract which had a dry residue of 1.6% by weight. After spray drying, a powder was obtained in a yield of 9.1% by weight, based on the dry weight.

Example 2. Example 1 was repeated except that extraction was carried out with a 1:1 mixture of methanol and water. After spray drying, a powder was obtained in a yield of 18.5% by weight, based on the dry weight.

Example 3. Example 1 was repeated using leaves of Spirobolus cubensis (Hitchcock). A powder was obtained in a yield of ca. 10% by weight, based on the dry weight.

Example 4. Example 1 was repeated using leaves of Selaginella lepidophylla. A powder was obtained in a yield of ca. 10% by weight, based on the dry weight.

Example 5. Example 1 was repeated using leaves of Xerophyta retinervis. A powder was obtained in a yield of ca. 10% by weight, based on the dry weight.

Example 6. Example 1 was repeated except that extraction was carried out with leaves of Craterostigma plantigineum using 300 ml 95% by weight ethanol. The leaves were extracted twice as described above and the extracts were combined. Thereafter, first the alcohol was removed under reduced pressure at 45° C. and then the residue was dried at 50° C. A powder was obtained in a yield of ca. 20% by weight, based on the dry weight of the leaves used.

Example 7. 1 kg fresh baker's yeast Saccharomyces cerevisiae was suspended in 2 liters water with 50 mM NaCl. The pH of the solution was adjusted to 7.5 with 2n NaOH, after which the solution was heated for 15 mins. at 100° C. and then cooled. The cells were destroyed at 800 bar in a discontinuous high-pressure homogenizer. The pH was adjusted to 4 with 2n sulfuric acid, after which the suspension was reheated for 15 mins. to 100° C. and then cooled. Insoluble fractions were removed by centrifuging for 30 mins. at 5600 G and the supernatant solution was filtered. The opalescent solution obtained was dried and 4.3% dry product were obtained.

### Example 8

Cell Protecting Effect against UVA on Human Fibroblasts Cultivated In Vitro

Background: UV-A rays penetrate into the dermis where they lead to oxidative stress which is demonstrated by lipoperoxidation of the cytoplasm membranes.

The lipoperoxides are degraded to malonaldialdehyde which will crosslink many biological molecules, such as proteins and nuclein bases (enzyme inhibition or mutagenesis).

Method: To carry out these tests, a defined culture medium (DMEM) containing the fibroblasts was inoculated with foetal calf serum and added to the plant extract (in the defined medium containing 10% foetal serum) 72 hours after inoculation. Incubration was carried out at 37° C./5% CO₂.

After incubation for 48 hours at 37° C./5% CO₂, the culture medium was replaced by saline solution (physioloigcal NaCl solution) and the fibroblasts were exposed to a dose of UVA (365 nm, 20 J/cm²; tubes: MAZDA FLUOR TFWN40).

After the exposure to UVA, the MDA level (malonaldialdehyde level) in the supernatant sodium chloride solution was quantitatively determined by reaction with thiobarbituric acid. The protein content was determined by Bradford's method using a Coomassie Brilliant Blue color (Bradford, Analytical Biochem., 72; 248-254; 1976).

TABLE 1
MDA level and protein content for determining the cell protecting
effect against UVA (the standard deviation is shown in brackets)

| | MDA level | Protein content |
|---|---|---|
| Concentration (% by weight) | [% versus control] | [% versus control] |
| Control without UV | 0 | 100 |
| UVA (365 nm) | 100 | 103(7) |
| UVA + vitamin E | 31(4) | 102(11) |
| UVA + extract 0.1% | 91(5) | 101(16) |
| UVA + extract 0.3% | 67(6) | 100(17) |

The results set out in Table 1 show that the extracts of the plant Myrothamnus flabellifolia significantly reduce the level of MDA in human fibroblasts which is induced by UVA radiation. These results reflect a high capacity to reduce the harmful effects of oxidative stress on the skin. The protein content again demonstrates the nontoxic effect of the extract.

### Example 9

Cell Protecting Effect against UVB on Human Keratinocytes Cultivated In Vitro

Method: the effect of UVB radiation was investigated in vitro on keratinocytes by determining the release of the cytoplasm enzyme LDH (lactate dehydrogenase). This enzyme serves as a marker for cell damage.

To carry out the test, a defined medium (DMEM) containing fetal calf serum was inoculated with the keratinocytes and added to the plant extract (diluted with saline solution) 72 hours after inoculation.

The keratinocytes were then exposed to a UVB dose (50 mJ/cm²—tubes: DUKE FL40E).

After incubation for another day ay 37° C./5% CO₂. the LDH content in the supernatant phase was determined. The LDH (lactate dehydrogenase) content was spectrophotometrically determined by determining the NADH content during the LDH-catalyzed conversion of pyruvate to lactate by Bonnekoh's method (Bonnekoh B. et al.; Dermatol. Research; 282; 325-329;1990).

The number of adhering keratinocytes was determined by a DNA assay based on the fluorescence measurement of fluorochromium that binds to cellular DNA using Desaulniers' method (Desaulni rs D. et al.; Toxicol. in vitro; 12; 409-422; 1998) and a particle counter. Another test was carried out for comparison using a standard anti-inflammatory, acetyl salicylic acid.

TABLE 2
Content of released LDH for determining the cell protecting effect
against UVB (the standard deviation is shown in brackets)

| Extract of Example 1 | Number of | Content of LDH |
|---|---|---|
| [% by weight] | keratinocytes | released |
| Control without UV | 100 | 0 |
| UVB (315 nm) | 25(3) | 100 |
| UVB + acetylsalicylic acid (0.03%) | 76(5) | 3(2) |
| UVB + extract 0.1% | 27(4) | 91(8) |
| UVB + extract 0.3% | 40(7) | 57(20) |

The results of these tests show that the extracts positively influence the effect of UVB radiation on the number of keratinocytes and on the content of released LDH in a concentration of 0.3% by weight. Accordingly, the described extracts have the ability to reduce the damage to cell membranes caused by UVB radiation.

### Example 10

Cell Protection against Heat Shock in Human Fibroblasts

The heat shock in human fibroblasts was induced by increasing the incubation temperature from 37° C. to 45° C. for two hours. The number of living stressed cells was determined through the content of cellular adenosine triphosphate (ATP) and lactate dehydrogenase (LDH). The ATP content is well-known marker of cellular viability and a modified content is a very sensitive test for cytotoxicity. The content was determined by Vasseur's method (Vasseur P. et al.; Environmental Pollution; 1; 167-175; 1980).

The release of the high molecular weight cytoplasm enzyme LDH is a sign of cell membrane damage and is a general marker for cell damage. The LDH (lactate dehydrogenase) content was spectrophotometrically determined by determining the NADH content during the LDH-catalyzed conversion of pyruvate to lactate by Bonnekoh's method (Bonnekoh B. et al.; Dermatol. Research; 282; 325-329;1990).

Method: To carry out these tests, a defined culture medium (DMEM) containing the fibroblasts was inoculated with fetal calf serum and added to the plant extract or to the mixtures and preparations to be tested (in the defined medium containing 10% fetal calf serum) 72 hours after inoculation. Incubation was carried out at 37° C./5% CO₂.

After incubation for 48 hours at 37° C./5% CO₂, the cells were exposed to the heat shock by increasing the incubation temperature from 37° C. to 45° C. for two hours. The cells were then re-incubated for 24 hours at 37° C./5% CO₂.

The ATP content was monitored by determining the light component in the enzymatic reaction between ATP and the complex of luciferin/luciferase.

In addition to the extract of Example 1, a mixture containing water, glycerol, trehalose, polysaccharides from Tamarindus indica seeds and Myrothamnus flabellifolia extract and a preparation containing the Myrothamnus flabellifolia extract of Example 1 and the yeast extract of Example 7 in a ratio of 1:1 were tested in a concentration of 0.01% by weight.

TABLE 3
Content of released LDH and released ATP for determining
the cell protecting effect against heat shock (the
standard deviation is shown in brackets)

| | ATP | Content of |
|---|---|---|
| | content | released |
| Treatment | [%] | LDH [%] |
| Control without heat shock | 100 | 0 |
| Control with heat shock | 17(5) | 100 |
| Extract of Example 1/0.3% by weight + | 77(4) | 16(7) |
| heat shock | | |
| Mixture/1% by weight + heat shock | 40(18) | 50(13) |
| Preparation of extract of Examples 1 and 7 | 74 | 8 |
| (ratio 1:1)/0.01% by weight + heat shock | | |

The harmful effect of heat shock on human fibroblasts was reflected in the reduced ATP content and the increased content of released LDH. The treatment with Myrothamnus flabellifolia extract resulted in cell resistance to heat shock. A concentration of 0.3% by weight virtually eliminated the harmful effect of heat shock as determined through the ATP content and the content of released LDH.

### Example 11

Cell Protection against Cold Shock in Human Lymph Cells

The viability of stressed cells was investigated in human lymph cells by a test with propidium iodide. Propidium iodide is not taken up into the cell by intact cells, i.e. it does not penetrate through the intact cell wall. Only cell damage allows the fluorescence marker to penetrate into the cell. Destroyed cells thus become fluorescent and the uptake of the marker can be quantified by flow cytometry (cf. Lemaster J. J. et al.; Nature, 325, 78-81, 1987).

Method: The lymph cells were cultivated for one day in a standard medium (RPMI 1640 Complete, a product of Sigma). The standard growth medium was then replaced by a medium which either served as control medium or contained the mixture to be tested of Example 10 containing water, glycerol, trehalose, polysaccharides from Tamarindus indica seeds and Myrothamnus flabellifolia extract and was incubated for another day. The cold shock was produced by deep freezing for 15 minutes at −20° C. The test results were determined by Lemaster's method of flow cytometry either after a 15-minute post-shock incubation at +20° C. or after 4 hours' incubation at 37° C. The values for lymph cells without cold shock and addition of the mixture (0.1% by weight) were determined after the same incubation times except that the cells were not exposed to the 15-minute cold shock.

TABLE 4
Content of fluorescent cells for determining the cell protecting effect
against cold shock (the standard deviation is shown in brackets)

Propidium iodide -
positive cells [%]

| | 15 mins. after | 4 h after |
|---|---|---|
| Treatment | cold shock | cold shock |
| LyC without cold shock | 11.2 (0.4) | 9.9 (0.17) |
| LyC + mixture/0.1% | 12.4 (0.23) | 6.7 (0.17) |
| by weight | | |
| LyC with cold shock | 15.2 (0.57) | 24.3 (6.75) |
| LyC + mixture/0.3% | 12 (1.09) | 11.2 (0.86) |
| by weight + cold shock | | |

LyC = lymph cells

A 15-minute cold shock period after incubation for 4 hours shows an increase in destroyed cells which have taken up the fluorescence marker. The viability of the lymph cells after a cold shock was significantly increased by the treatment with a mixture containing water, glycerol, trehalose, polysaccharides from Tamarindus indica seeds and Myrothamnus flabellifolia extract.

### Example 12

Cell Protection against Osmostic Stress in Red Blood Corpuscles (Erythrocytes)

Resistance to osmotic stress or even osmotic shock in terms of membrane-stabilizing activity was tested on human red blood corpuscles by contacting them with a hypo-osmotic medium.

Method: First a solution of buffered hypo-osmotic salt solution containing 0.24 g/l NaCl was prepared and the red blood corpuscles were incubated in that solution for 60 mins. at room temperature. The mixture to be tested containing water, glycerol, trehalose, polysaccharides from Tamarindus indica seeds and Myrothamnus flabellifolia extract was added in different concentrations. For control purposes, the cells were incubated without the mixture to be tested, but in the osmotic salt solution. The cells were then centrifuged for 10 mins. at 3000 r.p.m. The intensity of the hemolysis used (emergence of hemoglobin from the erythrocytes) was monitored spectrophotometrically at an optical density of 412 nm.

TABLE 5
Intensity of hemoglobin released for determining
the cell protecting effect against osmotic shock
(the standard deviation is shown in brackets)

| | Intensity of |
|---|---|
| Treatment | hemoglobin released |
| Control with osmostic shock | 100 |
| Mixture/1% by weight + osmotic shock | 91 (6) |
| Mixture/3% by weight + osmotic shock | 45 (6) |

The tests demonstrate the cell-protecting activity of the tested mixture containing water, glycerol, trehalose, polysaccharides from Tamarindus indica seeds and Myrothamnus flabelifolia extract against osmotic shock. This effect is significantly reflected in a reduced release of hemoglobin from the stressed erythrocytes at a concentration of 3% by weight of the solution.

### Example 13

Skin Moisture Regulating Test

Background: The epidermis of human skin contains the horny layer (the stratum corneum). The Stratum corneum is a dielectric medium of low electrical conductivity. The water content leads to an increase in the dielectrical conductivity so that determination of the dielectrical conductivity of the stratum corneum can serve as a measure of the moisture content of human skin. The increase in the dielectrical conductivity of the Stratum corneum reflects an increase in the moisture content of human skin.

Methods: Samples of normal skin obtained from plastic surgery were used for this test. The Stratum corneum from these skin samples was stored in chambers with defined relative moisture (44%, saturated potassium carbonate solution) and standardized. Each sample of the Stratum corneum was comparatively tested under four conditions, namely:

1. without treatment

2. treatment with placebo

3. treatment with a preparation consisting of a hydrogel (Hyrogel LS from Laboratoire Sérobiologique LS) containing 1.125% by weight of Myrothamnus flabelliflolia extract

4. treatment with a preparation consisting of a hydrogel (Hyrogel LS from Laboratoire Sérobiologique LS) containing 3% by weight of a mixture containing water, glycerol, trehalose, polysaccharides from Tamarindus indica seeds and Myrothamnus flabellifolia extract

The placebo was the hydrogel (Hydrogel LS from Laboratoire Sérobiologioque) without the described preparation, i.e. without plant extract.

The moisture-regulating activity of the above-described preparation was determined as a percentage increase in conductivity by comparison with the placebo treatment.

The results reflect a dose-dependent moisture-regulating activity.

TABLE 1
Moisture-regulating effect as determined by measurement of the dielectrical conductivity
(in μS); mean value of 18 tests (the standard deviation is shown in brackets)

| Type of | Before the | | | | | | |
|---|---|---|---|---|---|---|---|
| treatment | treatment | 30 mins. | 1 h | 2 h | 4 h | 6 h | 24 h |
| Control | 22.5 (4.4) | 22.1 (2.7) | 21.9 (3.8) | 23.9 (4.4) | 20.3 (3.3) | 22.9 (3.9) | 20.0 (3.0) |
| Placebo | 23.7 (1.8) | 59.1 (2.4) | 41.3 (2.7) | 34.9 (2.1) | 33.6 (2.4) | 33.0 (3.0) | 32.0 (1.7) |
| Treatment 3 | 22.9 (1.6) | 82.4 (11.9) | 50.7 (2.8) | 40.1 (3.0) | 35.1 (2.5) | 32.6 (2.4) | 31.8 (1.8) |
| Treatment 4 | 24.4 (2.8) | 111.1 (6.5) | 80.7 (4.2) | 67.9 (4.6) | 56.9 (4.3) | 53.5 (4.4) | 54.6 (4.7) |

### Example 14

In order to determine the polysaccharide composition, the extracts of Examples 1 and 2 were subjected to thin-layer chromatography.

Solvent: acetone/butanol/phosphate buffer pH 7=50:40:10 (v/v) Coloring: N1-(naphthyl)-ethylenediamine Dihydrochloride (100° C.; 10-15 mins.).

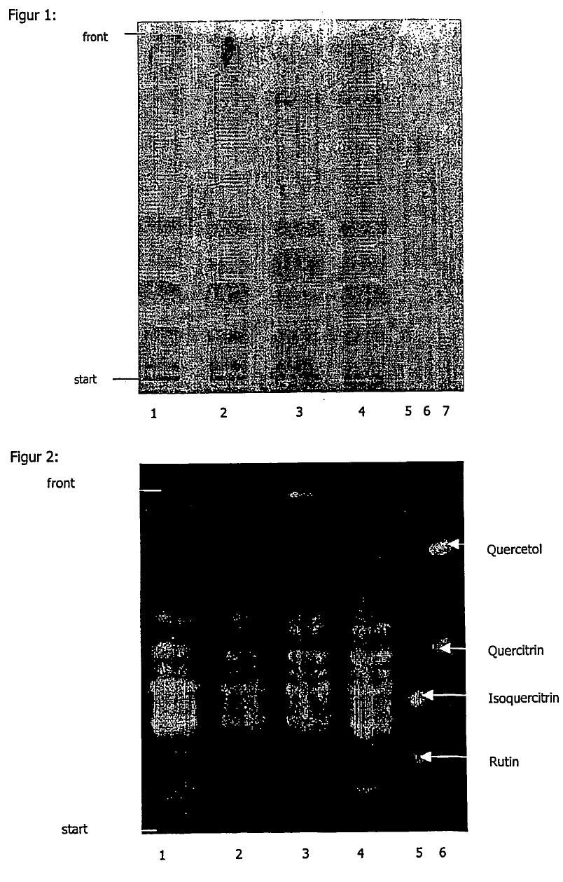

FIG. 1 shows the chromatogramm for Example 14.

The numbering under the chromatogram has the following meaning:

1: analytical extract of Myrothamnus flabellifolia

2: analytical extract of Myrothamnus flabellifolia

3: Myrothamnus flabellifolia extract of Example 1, 1% by weight

4: Myrothamnus flabellifolia extract of Example 2, 1% by weight

5: trehalose standard, 0. 1% by weight

6: rhamnose standard, 0.1% by weight

7: glucose standard, 0.1% by weight

### Example 15

In order to determine the radical trappers, the extracts of Examples 1 and 2 were subjected to further thin-layer chromatography.

Solvent: toluene/ethyl acetate/formic acid/water, 46:84:24:15 (v/v) Coloring: new+PEG (flavones), DMCA (tannins, anthocyans), 100° C., 10-15 mins.

FIG. 2 shows the chromatogram for Example 15. The numbering under the chromatogram has the following meaning:

1: Myrothamnus flabellifolia extract of Example 1, 1% by weight

2: Myrothamnus flabellifolia extract of Example 2, 1% by weight

3: 80% v/v analytical methanol extract, 7.5% v/v, Example 1

4: 80% v/v analytical methanol extract, 7.5% v/v, Example 2

5: standard mixture: rutin+isoquercetin

6: standard mixture: quercetin+quercetol

### Examples 16 and 17

Two so-called survival fractions prepared by thorough mixing of active constituents of the extracts according to the invention are reproduced in Table 7 below. Preparation was carried out by mixing the xyloglucans, the extracts and the glycerol at 70° C.; the other constituents were added later.

TABLE 7
Survival fractions

| Composition | Example 16 | Example 17 |
|---|---|---|
| Tamarind xyloglucans | 16.7 | 16.0 |
| Extract of Example 1 | 37.5 | — |
| Extract of Example 6 | — | 37.5 |
| Glycerol | 40.0 | 40.0 |
| Trehalose | 2.3 | 2.0 |
| Perservative | 3.5 | 3.5 |

| | |
|---|---|
| Water | to 100 |

The above results of the activity determination Examples show that the studied and tested Myrothamnus flabellifolia extracts have the following capabilities:

1. they reduce the degree of lipoperoxidation induced in human fibroblasts by UVA radiation.

2. they reduce the cell damage induced in human keratinocytes by UVB

3. they have a cell-protecting effect against heat shock, cold shock and osmotic shock and hence are active in protecting the skin against harmful environmental influences

4. a preparation containing extracts of the plant Myrothamnus flabellifolia showed clear moisture-regulating activity.

TABLE 2
Cosmetic preparations (water, preservative to 100% by weight)

| Composition (INCI) | 1 | 2 | 3 | 4 | 5 | 6 |
|---|---|---|---|---|---|---|
| Emulgade ® SE | 5.0 | 5.0 | 5.0 | 4.0 | — | — |
| Glyceryl Stearate (and) Ceteareth 12/20 | | | | | | |
| (and) Cetearyl Alcohol (and) Cetyl | | | | | | |
| Palmitate | | | | | | |
| Eumulgin ® B1 | — | — | — | 1.0 | — | — |
| Ceteareth-12 | | | | | | |
| Lameform ® TGI | — | — | — | — | 4.0 | — |
| Polyglyceryl-3 Isostearate | | | | | | |
| Dehymuls ® PGPH | — | — | — | — | — | 4.0 |
| Polyglyceryl-2 Dipolyhydroxystearate | | | | | | |
| Monomuls ® 90-O 18 | — | — | — | — | 2.0 | — |
| Glyceryl Oleate | | | | | | |
| Cetiol ® HE | — | — | — | — | — | 2.0 |
| PEG-& Glyceryl Cocoate | | | | | | |
| Cetiol ® OE | — | — | — | — | 5.0 | 6.0 |
| Dicaprylyl Ether | | | | | | |
| Cetiol ® PGL | — | — | — | 3.0 | 10.0 | 9.0 |
| Hexyldecanol (and) Hexyldecyl | | | | | | |
| Laurate | | | | | | |
| Cetiol ® SN | 3.0 | 3.0 | 3.0 | — | — | — |
| Cetearyl Isononanoate | | | | | | |
| Cetiol ® V | 3.0 | 3.0 | 3.0 | — | — | — |
| Deyl Oleate | | | | | | |
| Myritol ® 318 | — | — | — | 3.0 | 5.0 | 5.0 |
| Coco Caprylate Caprate | | | | | | |
| Bees Wax | — | — | — | — | 7.0 | 5.0 |
| Nutrilan ® Elastin E20 | 2.0 | 2.0 | — | — | — | — |
| Hydrolyzed Elasin | | | | | | |
| Extract of Example 1 | 0.1 | — | — | — | — | — |
| Extract of Example 2 | — | 0.1 | — | — | — | — |
| Extract of Example 3 | — | — | 0.1 | — | — | — |
| Extract of Example 4 | — | — | — | 0.1 | — | — |
| Extract of Example 5 | — | — | — | — | 0.1 | — |
| Extract of Example 6 | — | — | — | — | — | 0.1 |
| Nutrilan ® I-50 | — | — | 2.0 | — | — | — |
| Hydrolyzed Collagen | | | | | | |
| Gluadin ® AGP | — | — | — | 0.5 | — | — |
| Hydrolyzed Wheat Gluten | | | | | | |
| Gluadin ® WK | — | — | — | — | 0.5 | 0.5 |
| Sodium Cocoyl Hydrolyzed Wheat Protein | | | | | | |
| Highcareen ® | 1.0 | 1.0 | 1.0 | 1.0 | 1.0 | 1.0 |
| Betaglucan | | | | | | |
| Hydagen ® CMF | 1.0 | 1.0 | 1.0 | 1.0 | 1.0 | 1.0 |
| Chitosan | | | | | | |
| Magnesium Sulfate Hepta Hydrate | — | — | — | — | 1.0 | 1.0 |
| Glycerol (86% by weight) | 3.0 | 3.0 | 3.0 | 5.0 | 5.0 | 3.0 |
| Composition (INCI) | 7 | 8 | 9 | 10 | 11 | 12 |
| Emulgade ® SE | 5.0 | 5.0 | 5.0 | 4.0 | — | — |
| Glyceryl Stearate (and) Ceteareth 12/20 | | | | | | |
| (and) Cetearyl Alcohol (and) Cetyl | | | | | | |
| Palmitate | | | | | | |
| Eumulgin ® B1 | — | — | — | 1.0 | — | — |
| Ceteareth-12 | | | | | | |
| Lameform ® TGI | — | — | — | — | 4.0 | — |
| Polyglyceryl-3 Isostearate | | | | | | |
| Dehymuls ® PGPH | — | — | — | — | — | 4.0 |
| Polyglyceryl-2 Dipolyhydroxystearate | | | | | | |
| Monomuls ® 90-O 18 | — | — | — | — | 2.0 | — |
| Glyceryl Oleate | | | | | | |
| Cetiol ® HE | — | — | — | — | — | 2.0 |
| PEG-& Glyceryl Cocoate | | | | | | |
| Cetiol ® OE | — | — | — | — | 5.0 | 6.0 |
| Dicaprylyl Ether | | | | | | |
| Cetiol ® PGL | — | — | — | 3.0 | 10.0 | 9.0 |
| Hexyldecanol (and) Hexyldecyl Laurate | | | | | | |
| Cetiol ® SN | 3.0 | 3.0 | 3.0 | — | — | — |
| Cetearyl Isononanoate | | | | | | |
| Cetiol ® V | 3.0 | 3.0 | 3.0 | — | — | — |
| Deyl Oleate | | | | | | |
| Myritol ® 318 | — | — | — | 3.0 | 5.0 | 5.0 |
| Coco Caprylate Caprate | | | | | | |
| Bees Wax | — | — | — | — | 7.0 | 5.0 |
| Nutrilan ® Elastin E20 | 2.0 | 2.0 | — | — | — | — |
| Hydrolyzed Elastin | | | | | | |
| Preparation of Example 16 | 0.1 | — | — | — | — | — |
| Preparation of Example 17 | — | 0.1 | — | — | — | — |
| Octulose | — | — | 0.1 | — | — | — |
| Arbutin | — | — | — | 0.1 | — | — |
| Tamarind xyloglucans | — | — | — | — | 0.1 | — |
| Abscisic acid | — | — | — | — | — | 0.1 |
| Nutrilan ® I-50 | — | — | 2.0 | — | — | — |
| Hydrolyzed Collagen | | | | | | |
| Gluadin ® AGP | — | — | — | 0.5 | — | — |
| Hydrolyzed Wheat Gluten | | | | | | |
| Gluadin ® WK | — | — | — | — | 0.5 | 0.5 |
| Sodium Cocoyl Hydrolyzed Wheat Protein | | | | | | |
| Highcareen ® | 1.0 | 1.0 | 1.0 | 1.0 | 1.0 | 1.0 |
| Betaglucan | | | | | | |
| Hydagen ® CMF | 1.0 | 1.0 | 1.0 | 1.0 | 1.0 | 1.0 |
| Chitosan | | | | | | |
| Magnesium Sulfate Hepta Hydrate | — | — | — | — | 1.0 | 1.0 |
| Glycerol (86% by weight) | 3.0 | 3.0 | 3.0 | 5.0 | 5.0 | 3.0 |

(1, 2) Soft cream,
(3, 4) moisturizing emulsion,
(5, 6) night cream
(7, 8) Soft cream,
(9, 10) moisturizing emulsion,
(11, 12) night cream

1. Cosmetic and/or pharmaceutical preparations containing extracts of so-called resurrection plants.

2. Preparations as claimed in claim 1, characterized in that they contain extracts of resurrection plants selected from the botanical families Poacea, Scrophulariacea and Velloziacea.

3. Preparations as claimed in claims 1 and/or 2, characterized in that they contain extracts of resurrection plants selected from the group of botanical geni Myrothamnus, Sporobolus, Craterostigma, Xerophyta, Boeae, Ramonda, Haberlea, Chamaegigas and Selaginella.

4. Preparations as claimed in at least one of claims 1 to 4, characterized in that they contain extracts of protein-rich, angiospermous or gymnospermous plants or microorganisms.

5. Preparations as claimed in at least one of claims 1 to 3, characterized in that they contain extracts with effective contents of osmolytes, terpenes, antioxidants and/or phytohormones.

6. Preparations as claimed in claim 4, characterized in that they contain octulose, sucrose, glucose, trehalose, fructose, glycosyl-9-glycerol, xyloglucans and/or methylesters of pectin as the osmolytes.

7. Preparations as claimed in claim 4, characterized in that they contain carvone and/or perillic alcohol as the terpenes.

8. Preparations as claimed in claim 4, characterized in that they contain arbutin, anthocyans, superoxide dismustase, glutathione reductase and/or ascorbate peroxidase as the antioxidants.

9. Preparations as claimed in claim 4, characterized in that they contain abscisic acid as the phytohormone.

10. Preparations as claimed in at least one of claims 1 to 9, characterized in that they contain 0.001 to 1% by weight of the extracts, based on solids and the final preparation.

11. The use of extracts of resurrection plants for the production of cosmetic and/or pharmaceutical preparations.

12. The use of extracts of resurrection plants as active substances for regulating the water metabolism of the skin.

13. The use of extracts of resurrection plants as active substances for strengthening the cell metabolism as protection against harmful environmental influences.

14. The use of extracts of resurrection plants as active substances for protecting the skin and hair against damage by UV radiation.

15. The use of extracts of resurrection plants as active substances for protecting the skin and hair against free radicals.

16. The use of extracts of resurrection plants as active substances for protecting the macromolecules in the skin cells and cell membranes.

17. The use of octulose for the production of cosmetic and/or pharmaceutical preparations.

18. The use of arbutin for the production of cosmetic and/or pharmaceutical preparations.

19. The use of abscisic acid for the production of cosmetic and/or pharmaceutical preparations.
